(19) United States
(12) Patent Application Publication
(10) Pub. No.: US 2016/0051581 A1
(43) Pub. Date: Feb. 25, 2016

(54) DELIVERY OF ARGON TO A RECIPIENT OF BIOLOGICAL MATERIAL

(71) Applicant: L'AIR LIQUIDE SOCIETE ANONYME POUR L'ETUDE ET L'EXPLOITATION DES PROCEDES GEORGES CLAUDE, Paris (FR)

(72) Inventors: Ira KATZ, Meudon (FR); Marc LEMAIRE, Paris (FR); Jan PYPE, Herne (BE)

(73) Assignee: L'AIR LIQUIDE SOCIETE ANONYME POUR L'ETUDE ET L'EXPLOITATION DES PROCEDES GEORGES CLAUDE, Paris (FR)

(21) Appl. No.: 14/783,334

(22) PCT Filed: Apr. 9, 2014
(86) PCT No.: PCT/FR2014/050847

§ 371 Date: Oct. 8, 2015

(30) Foreign Application Priority Data
Apr. 12, 2013 (FR) 1353313

(51) Int. Cl. A61K 33/00; A61K 9/00

(52) U.S. Cl. CPC A61K 33/00; A61K 9/007

(57) ABSTRACT

The invention relates to a gaseous composition containing argon as an active gaseous compound for use to prevent or minimize an ischaemia-reperfusion lesion of a biological material in a recipient individual, during and/or subsequent to the transplantation of said biological material in said recipient individual, wherein said gaseous composition is administered to the recipient individual prior to, during and/or subsequent to the surgical operation aimed at grafting the biological material in said recipient individual.

### CROSS REFERENCE TO RELATED APPLICATIONS

This application is a 371 of International PCT Application PCT/FR2014/050847 filed Apr. 9, 2014 which claims priority to French Patent Application No. FR 1353313 filed Apr. 12, 2013, the entire contents of which are incorporated herein by reference.

### BACKGROUND

The present invention relates to a gas or gas mixture based on argon intended to be administered to a recipient of biological material, such as an organ, in particular a human being, prior to, during and/or after the surgical operation for transplanting said organ or said tissues, so as to protect said biological material before, during and/or after it is transplanted into the body of the recipient.

The withdrawal of biological material, that is to say of one or more organs and/or tissues, from an individual, referred to as donor, is a surgical act having a therapeutic purpose which results in a grafting, that is to say in the reimplantation of the biological material, referred to as graft, in another individual, referred to as recipient.

In order to avoid or minimize damage to this biological material, that is to say of these organs and/or tissues, it is necessary to protect them with specific solutions, referred to as preservation solutions, indeed even with specific gases, with which the organs and/or tissues are brought into contact after they have been withdrawn.

Thus, the document EP-A-2 536 272 teaches a liquid formulation comprising a liquid solution and at least one gas chosen from xenon, argon, hydrogen, H₂S, helium, krypton, neon, radon or CO. The gas is dissolved in the liquid “preservation” solution so as to preserve a biological material, in particular cells, tissues or biological organs, in particular an organ chosen from the heart, kidney, liver, pancreas and intestines. Preferably, the gas is argon.

In point of fact, between the moment of withdrawal and bringing the biological material into contact with the preservation medium, damage to said biological material may occur, in particular ischemia/reperfusion injuries, which are harmful to the biological material.

### DESCRIPTION OF PREFERRED EMBODIMENTS

The problem is consequently that of being able to prevent, reduce or minimize said ischemia/reperfusion injuries by implementing protection of the biological material, in particular of an organ or tissues, which is as effective as possible.

The solution of the invention is a gas composition comprising argon as active gas compound chosen for use in preventing or minimizing an ischemia/reperfusion injury to a biological material in a recipient individual, during and/or following the transplantation of said biological material in said recipient individual, said gas composition being administered to the recipient individual prior to, during and/or following the surgical operation targeted at grafting the biological material in said recipient individual.

As the case may be, the gas composition of the invention can comprise one or more of the following technical characteristics:
the biological material is an organ,
it comprises between 15 and 80% by volume of said active gas compound, that is to say of argon,
the biological material is a tissue, an organ or cells,
the biological material is an organ chosen from the liver, kidneys, lungs and heart,
the active gas compound is mixed with a gas comprising oxygen, prior to its administration to the recipient individual,
the recipient individual is a human being, that is to say a man, a woman, a child, a newborn, and the like,
it is administered in the gaseous form by inhalation,
it additionally comprises at least one additional active gas compound chosen from N₂, Xe, H₂, He, H₂S, Kr, Ne, NO and CO,
it optionally comprises oxygen,
it is composed of argon and of oxygen for the remainder, that is to say an Ar/O₂ gas mixture,
it is in the gaseous form and is administered by inhalation using a breathing mask or an intubation probe,
it comprises at least 20% by volume of said active gas compound,
it comprises at most 60% by volume of said active gas compound.

More generally, in the context of the present invention, it has been demonstrated that, in order to reduce damage or injuries resulting from ischemias/reperfusions in organs or other biological tissues withdrawn from a donor individual and intended to be reimplanted and grafted in a recipient individual, it is advisable to act, prior to, during and/or after the surgical operation targeted at grafting said organs or biological tissues in the body of the recipient, by administering a protective gas composition based on argon to the recipient.

In other words, a gas composition comprising a gas such as argon as a mixture with oxygen can be used in a therapeutic treatment method targeted at preventing or at minimizing an ischemia/reperfusion injury to a biological material in a recipient individual, such as a human being, during and/or following the transplantation of said biological material in said recipient individual, in which said gas composition is administered to the recipient individual prior to, during and/or following the surgical operation targeted at grafting the biological material in said recipient individual.

In addition, the invention relates to the use of argon for manufacturing a gas composition comprising argon as active gas compound for use in preventing or minimizing an ischemia/reperfusion injury to a biological material in a recipient individual, during and/or following the transplantation of said biological material in said recipient individual, said gas composition being administered to the recipient individual prior to, during and/or following the surgical operation targeted at grafting the biological material in said recipient individual.

The duration of administration of the gas can be easily determined on the basis of the duration of the anesthesia, via simple routine tests, as a function of the organ to be implanted and of the recipient individual considered.

### Illustrative Example

In order to show the effectiveness of a gas composition according to the invention formed of a gas mixture of argon and of oxygen in preventing or minimizing ischemia/reperfusion injuries to a biological material in a recipient individual, use is made of an animal model which makes it possible to demonstrate the effect of the administration of the gas, during and/or following the transplantation of said biological material in the recipient.

In this instance, the animals which are used for the tests are pigs and mice, anesthetized and intubated/ventilated, and the biological material is the lung.

In order to do this, a virtual withdrawal of the lungs from the animals is carried out by clamping said lungs, arteries and veins connected to the lungs so as to obtain complete exclusion of the lungs from the bodies of the test animals in a way identical to a real extraction.

Once clamping has been carried out, there is a wait for a period of approximately 90 minutes and then the lungs, veins and arteries are declamped.

The lungs of the animals are then ventilated for 4 hours with the test gas compositions.

The test composition (Ar/O₂) comprises contents of Ar of between 15 and 80% by volume, the remainder being oxygen.

A group of control animals is treated in the same way but with gas mixtures of nitrogen and of oxygen (N₂/O₂) in the same proportions.

The gas compositions are administered to the animals by inhalation, the animals being intubated and supplied with gas by a respiratory ventilator.

Subsequently, tests of the ventilatory function of the lungs are carried out.

The animals are then sacrificed and the lungs are extracted and subjected to anatomical studies in order to determine the ischemic state, that is to say the presence of possible injuries due to the ischemia/reperfusion period.

The results obtained show that the compositions according to the invention, based on argon, reduce ischemia/reperfusion injuries and their consequences on the satisfactory operation of the lungs, in contrast to the animals of the control group which have inhaled mixtures based on nitrogen.

These tests show the advantage in administering a gas composition according to the invention to a recipient individual, prior to, during and/or following the surgical operation targeted at grafting a biological material in said recipient individual, in particular an organ.

1.-10. (canceled)

11. A method of preventing or minimizing an ischemia/reperfusion injury to a biological material in a recipient individual, the method comprising the step of administering a gas composition comprising argon as active gas compound, prior to, during and/or following a surgical operation targeted at grafting the biological material into said recipient individual, to thereby prevent or minimize ischemia/reperfusion injury to the biological material during and/or following a transplantation of said biological material into said recipient individual.

12. The method of claim 11, wherein the gas composition comprises between 15 and 80% by volume of argon.

13. The method of claim 11, wherein the biological material is a tissue, an organ or cells.

14. The method of claim 11, wherein the biological material is an organ.

15. The method of claim 11, wherein the biological material is an organ chosen from the liver, kidneys, lungs and heart.

16. The method of claim 11, wherein the gas composition is mixed with a gas comprising oxygen, prior to its administration to the recipient individual.

17. The method of claim 11, wherein the recipient individual is a human being.

18. The method of claim 11, wherein the gas composition comprises at least 20% argon by volume and/or at most 60% argon by volume.

19. The method of claim 11, wherein the gas composition is administered in a gaseous form by inhalation.

20. The method of claim 11, wherein the gas composition is composed of argon and of oxygen.
